(19) United States
(12) Patent Application Publication
(10) Pub. No.: US 2017/0340298 A1
(43) Pub. Date: Nov. 30, 2017

(54) MOBILE BIPLANE FLUOROSCOPY APPARATUS WITH FOLDING ARM CONFIGURATIONS

(71) Applicant: Whale Imaging, Inc., Waltham, MA (US)

(72) Inventors: Changguo Ji, Lexington, MA (US); Li'an Yuan, Waltham, MA (US); Xingbai He, Belmont, MA (US); Xun Zhu, Waltham, MA (US)

(21) Appl. No.: 15/596,900

(22) Filed: May 16, 2017

Related U.S. Application Data
(60) Provisional application No. 62/392,321, filed on May 27, 2016.

(51) Int. Cl. A61B 6/00

(52) U.S. Cl. CPC A61B 6/4435; A61B 6/487; A61B 6/4007; A61B 6/4208; A61B 6/461

(57) ABSTRACT

A foldable biplane G-arm for use with x-ray imaging and fluoroscopic imaging. The system includes a gantry that supports imaging machinery to allow two bi-planar imaging beams to be taken simultaneously or without movement of the equipment and/or patient. Additionally, the system provides a G-arm gantry with a folding arm configuration that can solve prominent problems with traditional C-arm and G-arm gantries. In particular, the folding arm enables the G-arm of the present invention to reduce or eliminate obstacles between surgeons and nurses and more easily adapt for insertion of surgical tables into the system for imaging procedures by pivoting out and away from a center focus point of the support gantry to provide improved access for moving patient tables into and out of position for imaging the patient.

### CROSS-REFERENCE TO RELATED APPLICATION(S)

This application claims priority to, and the benefit of, co-pending U.S. Provisional Application No. 62/392,321, filed May 27, 2016, for all subject matter common to both applications. The disclosure of said provisional application is hereby incorporated by reference in its entirety.

### FIELD OF THE INVENTION

The present invention relates to a medical instrument suitable for use with bi-plane imaging (e.g., X-ray). In particular, the present invention relates to a mobile biplane G-arm configured with a folding arm enabling flexibly in the setup and operation of the G-arm in imaging applications.

### BACKGROUND

Generally, it is desirable to take X-rays of a patient from a number of different positions, preferably without the need for frequent repositioning of the patient. It is preferable that the X-ray apparatus not unduly encumber the space surrounding the patient to enable a physician to treat or otherwise attend to the patient without the need to repeatedly remove and replace the X-ray apparatus.

C-arm X-ray equipment has been developed to meet these needs and has become well known in the medical arts of surgical and other interventional procedures. An example of conventional C-arm equipment is shown in FIG. 1. A description of the C-arm equipment depicted in FIG. 1 is provided in U.S. Pat. No. 8,992,082, which is incorporated herein by reference. Generally, C-arm X-ray equipment is smart and flexible in operation, and in its positioning process, which can be deduced from the degrees of freedom of movement inherent in such a structure. Additionally, the C-arm gantry is typically mounted so as to enable rotational movement of the arm in two degrees of freedom. For example, a conventional C-arm gantry can be slidably mounted to the support structure to enable orbiting rotational movement of the C-arm about its center of curvature, represented in FIG. 1 as direction A. The C-arm equipment can also provide lateral rotation which is a motion rotating along the horizontal axis, represented in FIG. 1 as direction B. In addition, the C-arm equipment can also have an up-down motion along the vertical axis, represented in FIG. 1 as direction C, a cross-arm motion along the horizontal axis, represented in FIG. 1 as direction D, and a wig-wag motion along the vertical axis, represented in FIG. 1 as direction E.

However, this (technology, device, system, methodology, etc.) experiences some shortcomings. In particular, although the C-arm X-ray equipment is smart and flexible in its positioning capabilities, it is often desirable to take X-rays of a patient from multiple positions (e.g., bi-planar imaging), which can be difficult in such conventional setups. For example, when taking images from both the anteroposterior (AP) & lateral (LT) positions (two perpendicular angles), the operators have to reposition the C-arm because C-arm configurations do not allow for such perpendicular bi-planar imaging. In particular, C-arm systems are configured to capture a single plane image at a time, such that to get images from two different planes, the C-arm must capture an image in one plane, then be repositioned to capture an image in another plane.

In view of the shortcomings related to capturing bi-planar images with a traditional C-arm device, it is common for practitioners to utilize G-arm or G-shape arm equipment for taking images from different angles at the same time, without repositioning the imaging apparatus. In operation, when utilizing a G-arm or G-shaped arm, the two imaging beams (e.g., X-Rays) emitted from the two imaging sources (e.g., X-ray tubes) may cross at an iso-center. However, operation of the G-arm has its own shortcomings. Specifically, the bi-planar design of conventional G-arm equipment unduly encumbers a space surrounding the patient due to the 270 degree of mechanical angle of gantry coverage. This configuration creates obstacle between surgeon and nurses. Additionally, the G-arm can only adapt to a few surgical tables by slipping the gantry from their front, as reflected in the table positioning depicted in FIG. 1.

### SUMMARY

There is a need for a piece of equipment to enable bi-planar imaging without the shortcomings of traditional C-arm and G-arm configurations. The present invention is directed toward further solutions to address this need, in addition to having other desirable characteristics. Specifically, the present invention provides a mobile imaging apparatus that enables the advantages of both C-shaped, G-shaped, and ring-shaped arm configurations, without the shortcomings. The device of the present invention includes a gantry that supports imaging machinery (e.g., imaging energy emitters, imaging receptor, processing units, etc.). The gantry is formed to allow two bi-planar imaging beams to be taken simultaneously or without movement of the equipment and/or patient. Additionally, the present invention provides a G-arm gantry with a folding arm configuration can solve prominent problems with traditional C-arm and G-arm gantries. In particular, the folding arm enables to the G-arm of the present invention to reduce or eliminate obstacles between surgeons and nurses, more easily adapt for insertion of surgical tables, etc.

In accordance with example embodiments of the present invention, a foldable G-arm bi-planar imaging apparatus is provided. The apparatus includes a support gantry having a generally arc shape about an interior center focus point with a first terminal end and a second terminal end, the first terminal end being at a distal end of a folding arm coupled in an articulating manner with the support gantry at a pivot point. The apparatus also includes a first imaging assembly positioned on the support gantry, the first imaging assembly comprising a first imaging energy emitter positioned opposite a first imaging receptor, wherein one of the first imaging energy emitter and the first imaging receptor is positioned at the first terminal end of the support gantry on the folding arm. The apparatus further includes a second imaging assembly positioned on the support gantry, the second imaging assembly includes a second imaging energy emitter positioned opposite a second imaging receptor, wherein one of the second imaging energy emitter or the second imaging receptor is positioned at the second terminal end of the support gantry. The apparatus also includes a control unit that moves and positions the support gantry. The action of the folding arm articulating about the pivot point results in whichever of the first imaging energy emitter or the first imaging receptor that is positioned at the first terminal end of the support gantry on the folding arm articulating in a first direction out and away from, or in a second direction in and toward, the center focus point of the generally arc shape support gantry, commensurate with an articulating direction of the folding arm about the pivot point.

In accordance with aspects of the present invention, the first imaging assembly is positioned and oriented to emit imaging energy in an LT plane and the second imaging assembly is positioned and oriented to emit imaging energy in an AP plane, perpendicular to the LT plane. The first imaging assembly can be positioned and oriented to emit imaging energy in an AP plane and the second imaging assembly is positioned and oriented to emit imaging energy in an LT plane, perpendicular to the AP plane.

In accordance with aspects of the present invention, the action of the folding arm articulating about the pivot point in the direction out and away from the center focus point of the generally arc shape of the support gantry achieves a sufficiently low position to coincide with a height of a surgical table, wherein the first terminal end of the support gantry is foldable to enable rotation of the first terminal end low enough to adapt to a height of a surgical table. A pivot angle of the folding arm can ranges between an angle of 0 degrees to 180 degrees.

In accordance with aspects of the present invention, the first imaging receptor and the second imaging receptor are one of an image intensifier or a flat panel detector. The first imaging energy emitter and the second imaging energy emitter can be X-ray sources configured to produce X-ray beams.

In accordance with example embodiments of the present invention, a foldable G-arm bi-planar imaging system is provided. The system includes a support gantry having a generally arc shape about an interior center focus point with a first terminal end and a second terminal end, the first terminal end being at a distal end of a folding arm coupled in an articulating manner with the support gantry at a pivot point. The system also includes a first imaging assembly positioned on the support gantry, the first imaging assembly including a first imaging energy emitter positioned opposite a first imaging receptor, wherein one of the first imaging energy emitter and the first imaging receptor is positioned at the first terminal end of the support gantry on the folding arm. The system further includes a second imaging assembly positioned on the support gantry, the second imaging assembly including a second imaging energy emitter positioned opposite a second imaging receptor, wherein one of the second imaging energy emitter or the second imaging receptor is positioned at the second terminal end of the support gantry. The system The system includes a control unit that moves and positions the support gantry and a processing and display device in communication with the first imaging assembly and the second imaging assembly, the processing and display device configured to receive raw image data and display an image on a display device. The action of the folding arm articulating about the pivot point results in whichever of the first imaging energy emitter or the first imaging receptor that is positioned at the first terminal end of the support gantry on the folding arm articulating in a first direction out and away from, or in a second direction in and toward, the center focus point of the generally arc shape support gantry, commensurate with an articulating direction of the folding arm about the pivot point.

In accordance with aspects of the present invention, the first imaging assembly is positioned and oriented to emit imaging energy in an LT plane and the second imaging assembly is positioned and oriented to emit imaging energy in an AP plane, perpendicular to the LT plane. The first imaging assembly is positioned and oriented to emit imaging energy in an AP plane and the second imaging assembly can be positioned and oriented to emit imaging energy in an LT plane, perpendicular to the AP plane.

In accordance with aspects of the present invention, the action of the folding arm articulating about the pivot point in the direction out and away from the center focus point of the generally arc shape of the support gantry achieves a sufficiently low position to coincide with a height of a surgical table, wherein the first terminal end of the support gantry is foldable to enable rotation of the first terminal end low enough to adapt to a height of a surgical table. A pivot angle of the folding arm can range between an angle of 0 degrees to 180 degrees.

In accordance with aspects of the present invention, the first imaging receptor and the second imaging receptor are one of an image intensifier or a flat panel detector. The first imaging energy emitter and the second imaging energy emitter can be X-ray sources configured to produce X-ray beams.

In accordance with example embodiments of the present invention, a method for utilizing a foldable G-arm bi-planar imaging apparatus is provided. The method includes positioning, via a control unit, a support gantry. The apparatus includes a support gantry having a generally arc shape about an interior center focus point with a first terminal end and a second terminal end, the first terminal end being at a distal end of a folding arm coupled in an articulating manner with the support gantry at a pivot point. The apparatus also includes a first imaging assembly positioned on the support gantry, the first imaging assembly having a first imaging energy emitter positioned opposite a first imaging receptor, wherein one of the first imaging energy emitter and the first imaging receptor is positioned at the first terminal end of the support gantry on the folding arm. The apparatus further includes a second imaging assembly positioned on the support gantry, the second imaging assembly having a second imaging energy emitter positioned opposite a second imaging receptor, wherein one of the second imaging energy emitter or the second imaging receptor is positioned at the second terminal end of the support gantry. The method also includes disengaging a locking mechanism securing the folding arm. The method further includes articulating the folding arm about the pivot point such that whichever of the first imaging energy emitter or the first imaging receptor that is positioned at the first terminal end of the support gantry on the folding arm is articulated in a first direction out and away from, or in a second direction in and toward, the center focus point of the generally arc shape support gantry, commensurate with an articulating direction of the folding arm about the pivot point.

In accordance with aspects of the present invention, the articulation of the folding arm ranges between an angle of 0 degrees to 180 degrees. The articulation of the folding arm about the pivot point in the direction out and away from the center focus point of the generally arc shape of the support gantry can achieve a sufficiently low position to coincide with a height of a surgical table, such that the first terminal end of the support gantry is foldable to enable rotation of the first terminal end low enough to adapt to a height of a surgical table.

In accordance with aspects of the present invention, the method can include articulating the folding arm in the second direction in and toward the center focus point of the generally arc shape support gantry and engaging the locking mechanism securing the folding arm in preparation for operation of the apparatus.

### BRIEF DESCRIPTION OF THE FIGURES

These and other characteristics of the present invention will be more fully understood by reference to the following detailed description in conjunction with the attached drawings, in which:

FIG. 1 is an illustration of an example structure for a conventional C-arm as known in the art; and

FIGS. 2A, 2B, and 2C are diagrammatic illustrations of a G-arm apparatus, in accordance with the present invention.

### DETAILED DESCRIPTION

An illustrative embodiment of the present invention relates to a G-arm imaging device with a folding arm configured to rotate a portion of the G-arm gantry down and away from the center of the G-arm gantry, enabling better access. In particular, the present invention relates to a G-arm imaging device with a folding arm located between the six o'clock and the nine o'clock position of the G-arm gantry. The folding arm is configured to fold/rotate an end portion of the gantry, enabling improved access and imaging functionality. The folding arm is configured such that the energy emitter or imaging receptor attached to the gantry at the nine o'clock position is rotatable down and away from the central point of the gantry about the axis of the folding arm.

The G-arm gantry configured with the folding arm of the present invention enables a user of the G-arm apparatus to have more flexibility and comfort when operating the G-arm. In particular, the folding arm enables a user to have more flexibility when moving the G-arm gantry by reducing obstacles and obstructions caused by convention G-arm constructions. Additionally, the folding arm provides users with the flexibility with respect positioning a surgical table in and around the G-arm gantry. For example, the folding arm can be articulated down and away from the center point of the G-arm gantry to enable surgical tables of varied sizes to be utilized with the G-arm apparatus. Similarly, the folding arm can also provide a user (e.g., a surgeon) with more measurable surgical space if needed.

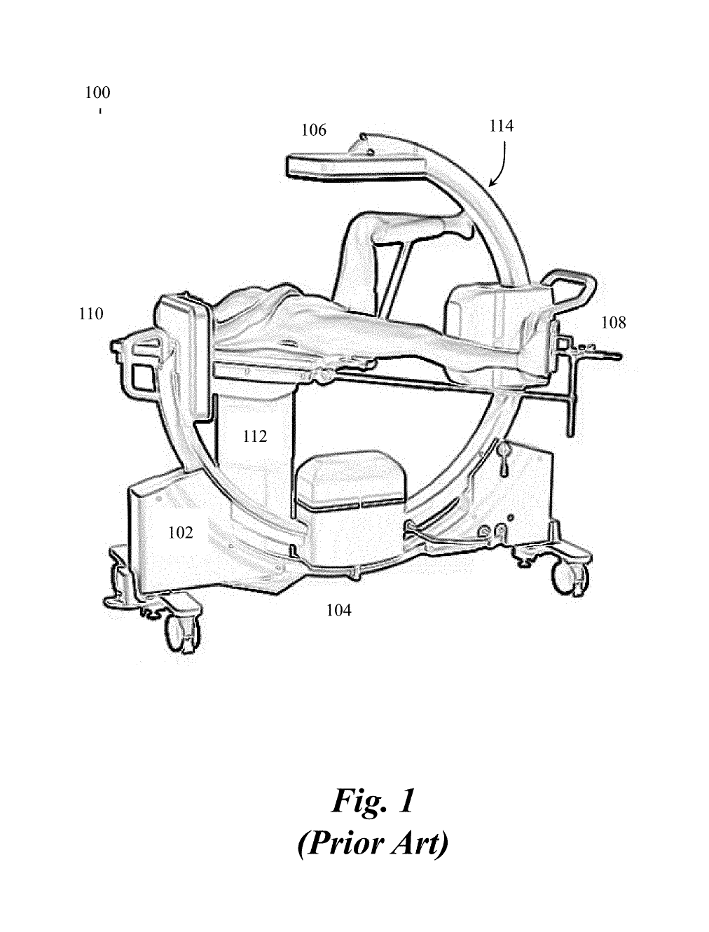

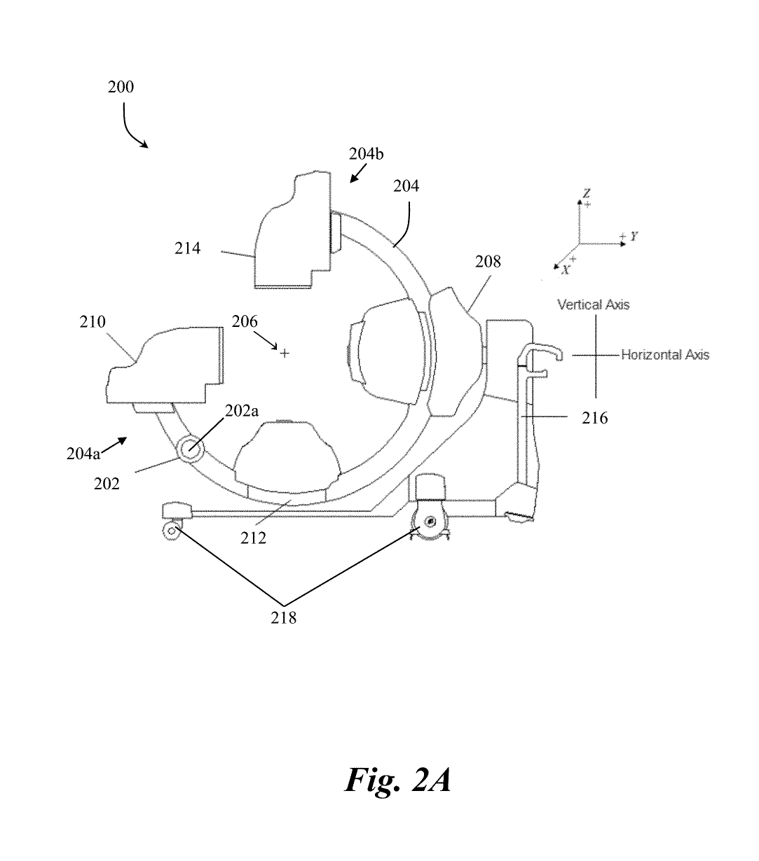

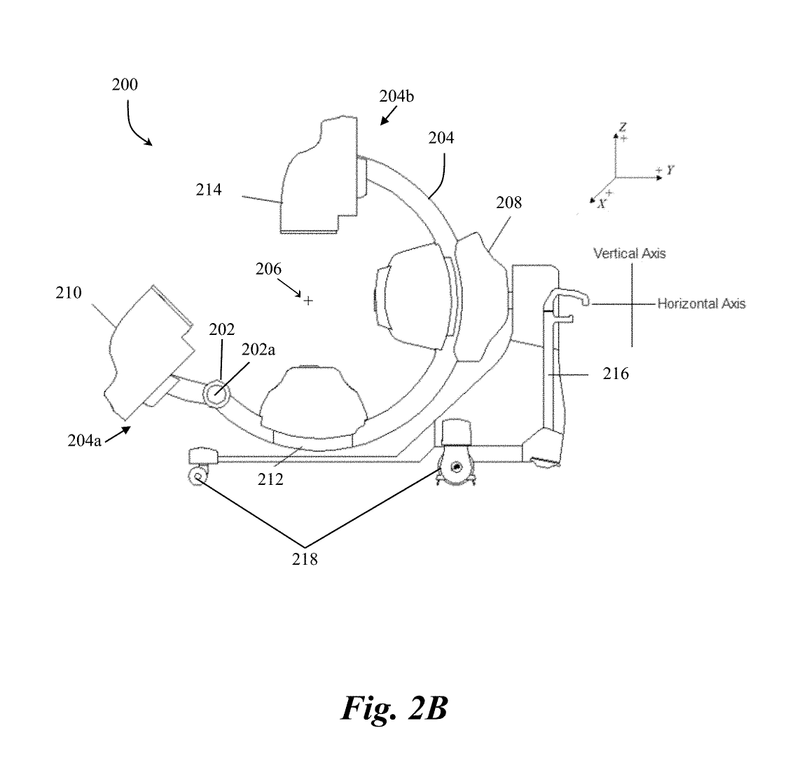

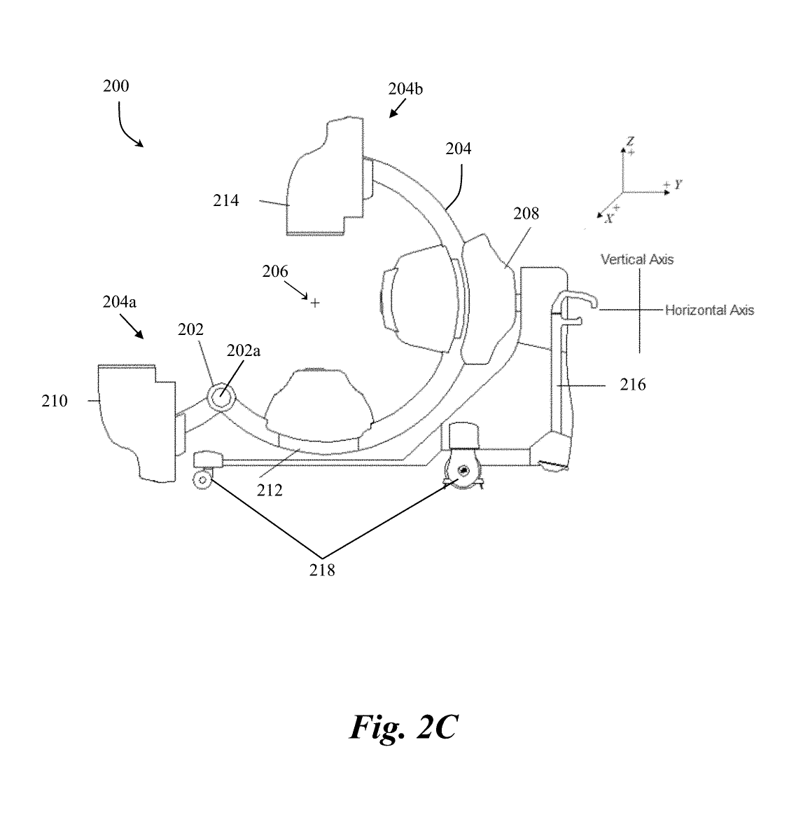

FIGS. 1 through 2C, wherein like parts are designated by like reference numerals throughout, illustrate an example embodiment or embodiments of an imaging device with a G-arm configuration and including a folding arm portion, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

As utilized herein, the phrase “LT plane” refers to the mean or sagittal plane of a patient, and the phrase “AP plane” refers to the transverse or axial plane of a patient, which is perpendicular to the LT plane. Such terminology is utilized in compliance with conventional meanings in the field of medical imaging.

Imaging systems are commonly utilized in the medical field and come in a variety of configurations for a variety of applications (e.g., C-arm single plane imager, G-arm bi-plane imager, etc.). An example of an imaging system configured for capturing bi-planar medical images (e.g., X-rays) of a patient is depicted in FIG. 1. In particular, FIG. 1 depicts a conventional G-arm medical imaging system 100 and the main components that make up the G-arm medical imaging system 100. The main components of the G-arm medical imaging system 100 system include a movable stand 102, a imaging energy emitter 104 (e.g., an X-ray source, X-ray tube, etc.) and imaging receptor 106 (e.g., an image intensifier, flat panel detector, etc.) configured for a frontal view (or anteroposterior view), an imaging energy emitter 108 and imaging receptor 110 configured for a lateral view, and a patient table 112 configured to hold a patient between the imaging energy emitters 104, 108 and the imaging receptor 106, 110. As would be appreciated by one skilled in the art, the imaging energy emitters 104, 108 can include any kind of suitable imaging energy emitters utilized for imaging a patient. For example, the imaging energy emitters 104, 108 can be electromagnetic radiation or x-imaging energy emitters configured to produce X-rays. The combination of elements in the G-arm medical imaging system 100 includes a gantry 114 that supports all of the components/machinery. The gantry 114 of the G-arm medical imaging system 100 is formed to allow two bi-planar images to be captured simultaneously or without movement of the equipment and/or the patient. In some instances, the gantry 114 is adjustable to change angles of the imaging machinery (e.g., the imaging energy emitters 104, 108 and imaging receptor 106, 110). Additionally, in some implementations, the imaging receptor 106, 110 portion of the G-arm medical imaging system 100 can be positioned on retractable and extendable arms, allowing the apparatus to have a larger access opening when not in operation, but to still provide bi-planar imaging capability during operation of the G-arm medical imaging system 100. However, even with such retractable and extendable arms, the conventional G-arm system still requires patient tables 112 to enter the imaging area by longitudinally positioning the patient table 112 end-first along an axis normal to the center-point of the circular cross-sectional area having a perimeter of the G-arm gantry 114, making access limited. More specifically, conventional G-arm systems are only configured to adapt to a surgical table with one end suspended; whereas the G-arm medical imaging system 100 of the present invention enables a patient table 112 to pass laterally over the G-arm gantry 114 (said differently, the G-arm gantry 114 can be pass through the patient table 112, underneath the horizontal surface upon which the patient rests), without requiring positioning the patient table 112 longitudinally through the normal axis to the center-point of the circular cross-sectional area.

FIGS. 2A-2C depict example illustrations of an imaging apparatus 200 for use in accordance with the present invention. In particular, FIGS. 2A-2C depict different orientations of a G-arm bi-planar imaging apparatus 200 including a folding arm 202. More specifically, FIG. 2A depicts the apparatus 200 with the folding arm 202 in an unfolded position, FIG. 2B depicts the apparatus 200 with the folding arm 202 in a partially folded position (e.g., between the unfolded position of FIG. 2A and the folded position in FIG. 2C), and FIG. 2C depicts the apparatus 200 with the folding arm 202 in a fully folded position. With respect to the particular type of operation for each of the configurations depicted in FIGS. 2A-2C, FIG. 2A depicts the apparatus 200 with the folding arm 202 in an imaging operational position (e.g., the positioning for operation of the apparatus 200 for performing imaging functions), FIG. 2B depicts the apparatus 200 with the folding arm transitioning between the operational position in FIG. 2A and a loading position in FIG. 2C, and FIG. 2C depicts the apparatus 200 with the folding arm 202 folded down to a loading position (e.g., enabling a user to position a patent table 112 about a center focus point 206 of the apparatus 200).

FIGS. 2A-2C each depict the same apparatus 200 with the same components arranged in varied orientations for different modes of operation provided by the folding arm 202. Accordingly, the components of the apparatus 200 are discussed with respect to each of the FIGS. 2A-2C, unless otherwise specified. In accordance with an example embodiment of the present invention, the apparatus 200 includes a support gantry 204 having a generally arc shape, about an interior center focus point 206, with a first terminal end 204a and a second terminal end 204b, the first terminal end 204a being at a distal end of the folding arm 202. The folding arm 202 is coupled in an articulating manner with the support gantry 204 at a pivot point 202a. In accordance with an example embodiment of the present invention, the folding arm 202 is motorized to rotate from an operational state (as depicted in FIG. 2A) to an accessible state (as depicted in FIG. 2C). As would be appreciated by one skilled in the art, the folding arm 202 can be implemented utilizing any mechanism to enable the end portion of the gantry to rotate as depicted in FIGS. 2A-2C. For example, the folding arm 202 can be implemented using a motorized pivotal axis implementing a flexible wiring connection to enable the foldable range of motion discussed herein.

The apparatus 200 also includes a first imaging assembly positioned on the support gantry 204, the first imaging assembly includes a first imaging energy emitter 208 positioned opposite a first imaging receptor 210. In accordance with an example embodiment of the present invention, one of the first imaging energy emitter 208 and the first imaging receptor 210 is positioned at the first terminal end 204a of the support gantry on the folding arm 202. The first imaging assembly is positioned and oriented, as depicted in FIGS. 2A-2C, to emit imaging energy (e.g., from the energy emitter 208) in an LT plane. FIGS. 2A-2C depict the first imaging receptor 210 at the first terminal end 204a of the support gantry 204, however as would be appreciated by one skilled in the art, the first imaging energy emitter 208 could be positioned at the first terminal end 204a with the first imaging receptor 210 positioned on the opposite side of the gantry 204 without influencing the imaging process. In other words, the imaging receptor 210 (shown in FIGS. 2A-2C) can be switched with the first imaging energy emitter 208 positionally (shown in FIGS. 2A-2C). Additionally, as would be appreciated by one skilled in the art, the first imaging assembly can alternatively be positioned and oriented to emit imaging energy (e.g., from the energy emitter 212) in an AP plane.

Continuing with FIGS. 2A-2C, the apparatus 200 further includes a second imaging assembly positioned on the support gantry 204, the second imaging assembly including a second imaging energy emitter 212 positioned opposite a second imaging receptor 214. In accordance with an example embodiment of the present invention, one of the second imaging energy emitter or the second imaging receptor is positioned at the second terminal end 204b of the support gantry 204. The second imaging assembly is positioned and oriented, as depicted in FIGS. 2A-2C, to emit imaging energy in an AP plane, perpendicular to the LT plane created by the first imaging assembly. FIGS. 2A-2C depicts the second imaging receptor 214 at the second terminal end 204b of the support gantry 204, however as would be appreciated by one skilled in the art, the second imaging energy emitter 212 could be positioned at the second terminal end 204b with the second imaging receptor 214 positioned on the opposite side of the gantry 204 without influencing the imaging process. In other words, the second imaging receptor 214 (shown in FIGS. 2A-2C) can also be switched with the second imaging energy emitter 212 (shown in FIGS. 2A-2C) in an optional arrangement. Additionally, as would be appreciated by one skilled in the art, the second imaging assembly can be positioned and oriented to emit imaging energy in an LT plane, perpendicular to the AP plane from the first imaging assembly.

The apparatus 200 also includes a control unit 216 configured to move and position the support gantry 204 at a desired location. In accordance with an example embodiment of the present invention, the support gantry 204 includes a plurality of wheels 218 to enable a user to push, pull, and pivot the apparatus 200 into a desired position via the control unit 216.

In operation, the folding arm 202 can be folded by rotating the folding arm 202 about an X-axis, about the pivot point 202a, as depicted in FIGS. 2A-2C. In accordance with an example embodiment of the present invention, the action of the folding arm 202 articulates about the pivot point 202a and results in whichever of the first imaging energy emitter 208 or the first imaging receptor 210 (as depicted in FIGS. 2A-2C) that is positioned at the first terminal end 204a of the support gantry 204 on the folding arm 202 articulating. The articulating motion occurs in a first direction out and away from, or in a second direction in and toward, the center focus point 206 of the generally arc shape support gantry 204, commensurate with the articulating direction of the folding arm 202 about the pivot point 202a, as depicted in FIGS. 2A-2C. By articulating the folding arm 202 out and away from the center focus point 206 to the loading position depicted in FIG. 2C, the folding arm 202 provides more operating space to eliminate the obstacles between surgeon and nurses under some circumstances and provides easy setup of a patient table to be inserted through the gap created by the articulated folding arm 202. In particular, the action of the folding arm 202 articulating about the pivot point 202a in the direction out and away from the center focus point 206 of the arc shape of the support gantry 204 achieves a sufficiently low position to relative to a height of a surgical table, such that the end portion of the first terminal end 204a of the support gantry 204 is foldable to enable rotation of the end portion low enough to adapt to, meaning being just below, a height of an underside of conventional surgical tables. For example, when a user desires to move a surgical table into the G-Arm apparatus 200, the user can rotate the folding arm 202 to partially or fully folded position, depending on the height of surgical tables, and insert the table above and across the folded down folding arm 202. Additionally, the G-arm apparatus 200 with the folding arm 202 can adapt to a surgical table such that the G-arm can be positioned (e.g., pushed) underneath the table and is not limited to surgical tables that are only suspended from a single end. In accordance with an example embodiment of the present invention, folding arm 202 includes a rotational joint at the pivot point 206 with a pivot angle ranging between an angles of 0 degrees to 180 degrees.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about” and “approximately” are intended to cover variations that may existing in the upper and lower limits of the ranges of subjective or objective values, such as variations in properties, parameters, sizes, and dimensions. In one non-limiting example, the terms “about” and “approximately” mean at, or plus 10 percent or less, or minus 10 percent or less. In one non-limiting example, the terms “about” and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A foldable G-arm bi-planar imaging apparatus, the apparatus comprising:
a support gantry having a generally arc shape about an interior center focus point with a first terminal end and a second terminal end, the first terminal end being at a distal end of a folding arm coupled in an articulating manner with the support gantry at a pivot point;
a first imaging assembly positioned on the support gantry, the first imaging assembly comprising a first imaging energy emitter positioned opposite a first imaging receptor, wherein one of the first imaging energy emitter and the first imaging receptor is positioned at the first terminal end of the support gantry on the folding arm;
a second imaging assembly positioned on the support gantry, the second imaging assembly comprising a second imaging energy emitter positioned opposite a second imaging receptor, wherein one of the second imaging energy emitter or the second imaging receptor is positioned at the second terminal end of the support gantry; and
a control unit that moves and positions the support gantry;
wherein action of the folding arm articulating about the pivot point results in whichever of the first imaging energy emitter or the first imaging receptor that is positioned at the first terminal end of the support gantry on the folding arm articulating in a first direction out and away from, or in a second direction in and toward, the center focus point of the generally arc shape support gantry, commensurate with an articulating direction of the folding arm about the pivot point.

2. The apparatus of claim 1, wherein the first imaging assembly is positioned and oriented to emit imaging energy in an LT plane and the second imaging assembly is positioned and oriented to emit imaging energy in an AP plane, perpendicular to the LT plane.

3. The apparatus of claim 1, wherein the first imaging assembly is positioned and oriented to emit imaging energy in an AP plane and the second imaging assembly is positioned and oriented to emit imaging energy in an LT plane, perpendicular to the AP plane.

4. The apparatus of claim 1, wherein the action of the folding arm articulating about the pivot point in the direction out and away from the center focus point of the generally arc shape of the support gantry achieves a sufficiently low position to coincide with a height of a surgical table, wherein the first terminal end of the support gantry is foldable to enable rotation of the first terminal end low enough to adapt to a height of a surgical table.

5. The apparatus of claim 1, wherein a pivot angle of the folding arm ranges between an angle of 0 degrees to 180 degrees.

6. The apparatus of claim 1, wherein the first imaging receptor and the second imaging receptor are one of an image intensifier or a flat panel detector.

7. The apparatus of claim 1, wherein the first imaging energy emitter and the second imaging energy emitter are X-ray sources configured to produce X-ray beams.

8. A foldable G-arm bi-planar imaging system, the system comprising:
a support gantry having a generally arc shape about an interior center focus point with a first terminal end and a second terminal end, the first terminal end being at a distal end of a folding arm coupled in an articulating manner with the support gantry at a pivot point;
a first imaging assembly positioned on the support gantry, the first imaging assembly comprising a first imaging energy emitter positioned opposite a first imaging receptor, wherein one of the first imaging energy emitter and the first imaging receptor is positioned at the first terminal end of the support gantry on the folding arm;
a second imaging assembly positioned on the support gantry, the second imaging assembly comprising a second imaging energy emitter positioned opposite a second imaging receptor, wherein one of the second imaging energy emitter or the second imaging receptor is positioned at the second terminal end of the support gantry;
a control unit that moves and positions the support gantry; and
a processing and display device in communication with the first imaging assembly and the second imaging assembly, the processing and display device configured to receive raw image data and displays an image on a display device;
wherein action of the folding arm articulating about the pivot point results in whichever of the first imaging energy emitter or the first imaging receptor that is positioned at the first terminal end of the support gantry on the folding arm articulating in a first direction out and away from, or in a second direction in and toward, the center focus point of the generally arc shape support gantry, commensurate with an articulating direction of the folding arm about the pivot point.

9. The system of claim 8, wherein the first imaging assembly is positioned and oriented to emit imaging energy in an LT plane and the second imaging assembly is positioned and oriented to emit imaging energy in an AP plane, perpendicular to the LT plane.

10. The system of claim 8, wherein the first imaging assembly is positioned and oriented to emit imaging energy in an AP plane and the second imaging assembly is positioned and oriented to emit imaging energy in an LT plane, perpendicular to the AP plane.

11. The system of claim 8, wherein the action of the folding arm articulating about the pivot point in the direction out and away from the center focus point of the generally arc shape of the support gantry achieves a sufficiently low position to coincide with a height of a surgical table, wherein the first terminal end of the support gantry is foldable to enable rotation of the first terminal end low enough to adapt to a height of a surgical table.

12. The system of claim 8, wherein a pivot angle of the folding arm ranges between an angle of 0 degrees to 180 degrees.

13. The system of claim 8, wherein the first imaging receptor and the second imaging receptor are one of an image intensifier or a flat panel detector.

14. The system of claim 8, wherein the first imaging energy emitter and the second imaging energy emitter are X-ray sources configured to produce X-ray beams.

15. A method for utilizing a foldable G-arm bi-planar imaging apparatus, the method comprising:
positioning, via a control unit, a support gantry, the apparatus comprising:
a support gantry having a generally arc shape about an interior center focus point with a first terminal end and a second terminal end, the first terminal end being at a distal end of a folding arm coupled in an articulating manner with the support gantry at a pivot point;
a first imaging assembly positioned on the support gantry, the first imaging assembly comprising a first imaging energy emitter positioned opposite a first imaging receptor, wherein one of the first imaging energy emitter and the first imaging receptor is positioned at the first terminal end of the support gantry on the folding arm; and
a second imaging assembly positioned on the support gantry, the second imaging assembly comprising a second imaging energy emitter positioned opposite a second imaging receptor, wherein one of the second imaging energy emitter or the second imaging receptor is positioned at the second terminal end of the support gantry;
disengaging a locking mechanism securing the folding arm; and
articulating the folding arm about the pivot point such that whichever of the first imaging energy emitter or the first imaging receptor that is positioned at the first terminal end of the support gantry on the folding arm is articulated in a first direction out and away from, or in a second direction in and toward, the center focus point of the generally arc shape support gantry, commensurate with an articulating direction of the folding arm about the pivot point.

16. The method of claim 15, wherein the articulation of the folding arm ranges between an angle of 0 degrees to 180 degrees.

17. The method of claim 15, wherein the articulation of the folding arm about the pivot point in the direction out and away from the center focus point of the generally arc shape of the support gantry achieves a sufficiently low position to coincide with a height of a surgical table, wherein the first terminal end of the support gantry is foldable to enable rotation of the first terminal end low enough to adapt to a height of a surgical table.

18. The method of claim 17, further comprising:
articulating the folding arm in the second direction in and toward the center focus point of the generally arc shape support gantry; and
engaging the locking mechanism securing the folding arm in preparation for operation of the apparatus.
